(19) United States
(12) Patent Application Publication
(10) Pub. No.: US 2025/0381046 A1
(43) Pub. Date: Dec. 18, 2025

(54) Artificial Intelligence Enabled Neuroprosthetic Hand

(71) Applicant: Regents of University of Minneostoa, Minneapolis, MN (US)

(72) Inventors: Zhi Yang, Minneapolis, MN (US); Anh Nguyen, Minneapolis, MN (US); Diu Khue Luu, Minneapolis, MN (US)

(21) Appl. No.: 18/711,707

(22) PCT Filed: Sep. 19, 2022
(86) PCT No.: PCT/US2022/044042

§ 371 Date: May 20, 2024

Related U.S. Application Data
(63) Continuation of application No. 17/948,175, filed on Sep. 19, 2022.
(60) Provisional application No. 63/245,903, filed on Sep. 19, 2021.
(60) Provisional application No. 63/245,903, filed on Sep. 19, 2021.

(51) Int. Cl. A61F 2/72; A61F 2/54; A61F 2/58; A61F 2/68; A61F 2/70; A61F 2/80; G05B 13/02

(52) U.S. Cl. CPC A61F 2/72; A61F 2/583; A61F 2/585; A61F 2/586; G05B 13/027; A61F 2002/543; A61F 2002/587; A61F 2002/6827; A61F 2002/704; A61F 2/80

(57) ABSTRACT

A prosthetic limb in amputation rehabilitation, having a forearm and a hand with four fingers and a thumb, with the wrist and the fingers & thumb thereof being fully independently controlled by nerve signals originating in the amputee's brain and not being controlled by the actions of nearby muscles in the amputee's upper arm or shoulder. Control of the prosthesis is achieved by a fully contained electronic unit in the forearm of the prosthesis that receives neural signals from the brain, converts the analog neural signals to digital signals that are fed into an artificial intelligence engine circuit that utilizes a library of algorithms to learn from the brain what the signals are that will produce a desired hand and finger movement, then convert its computed digital output to analog electrical signals that are fed to the prosthetic hand and finger to produce actual motion as instructed by the brain.

### CROSS-REFERENCE TO RELATED APPLICATIONS

This application is based on, claims priority to, and incorporates by reference in its entirety for all purposes, U.S. Provisional Application Ser. No. 63/245,903, filed Sep. 19, 2021, and entitled, “ARTIFICIAL INTELLIGENCE ENABLED NEUROPROSTHETIC HAND”.

### FIELD OF THE INVENTION

This invention generally concerns systems and methods for neuromodulation, and more specifically, a neuroprosthetic hand whose performance is improved by the use of deep learning artificial intelligence to control the motions of the hand precisely as the wearer intends.

### BACKGROUND OF THE INVENTION

General Background Electrical stimulation has been used for probing neural circuitry and identifying networks of neurons for many years. Despite its extensive use, the mechanism of electrical stimulation on the nervous system remains poorly understood, and the results of applying novel approaches to such therapeutic stimulation cannot be predicted due to the uncertainty of the outcome prior to experimental research and development. There exists a need for an integrated hardware and software platform system that can accurately and precisely sense, analyze, and modulate neural signals received directly from the human brain and transmitted therefrom to the peripheral nerves of the arm, or the leg in order to control the movements of the wrist, hand, and fingers of a neuroprosthesis am and hand, or the ankle, foot and toes of a neuroprosthesis leg with a latent time lag approximating the time taken for a nerve signal to travel from the brain to the limb. Currently available or known electrical stimulation devices do not adequately sense neural signals, process them in a time frame approximating that of a native system, and still comprise components that allow a wearer thereof to experience an acceptably natural, normal, range of motion and freedom from bulky designs.

Integrated Development Strategy. An integrated approach for developing a superior neuromodulation device will comprise the four strategies of developing ultra-technology for sensing neural signals, most preferably (but not exclusively) without requiring electrode penetration into a nerve; developing hardware technology for both selective amplification of signals of interest and for removing stimulation signal residual charges and noise artifacts to support simultaneous recording and stimulation of a nerve; developing a deep learning artificial intelligence neural network-based algorithm to analyze and decode signals; and developing prediction models of therapeutic outcomes, based on stimulation parameters and neural signals. The device of this present invention will, in its most preferred embodiment, address these four strategies of neuroprosthetic hand development, operating within the overall architecture of this four-part integrated approach. Such a strategy is needed to address the multiple inherent challenges in this field. Such challenges include: (1) the inability to harness the full range of movements of currently available dexterous prosthetic systems, for example the prior art DEKA Arm, the APL Arm, and the DLR Hand Arm system, each of which are described and cited below; (2) the efficacy of deep learning comes at the cost of computational complexity; (3) the inefficiency of conventional central processing units (CPU) that are found on most low-power platforms; (3) prior art deep learning models must be trained and deployed using graphics processing units (GPU) which have hundreds to thousands of multi-threaded computing units specialized for parallelized floating-point matrix multiplication; and (4) the state of current software support, i.e. prior art edge computing devices (devices that are at or near to the user) are compact (as hardware), and are suitable for deep learning uses, but current software is limited to highly customized neural networks, which hinders the full potential of, for example, a preferred embodiment of the invention as claimed, i.e. neural decoder implementation based on recurrent neural network (RNN).

Poor signal-to-noise ratio (SNR) of neural signals in nerves. Prior art neuroprostheses are not able to satisfactorily overcome the fact that nerve neural signals are smaller than signals measured in the brain, meaning that existing recording technology is inadequate for resolving nerve neural signals from sources of noise. Noise, in the context here of signal processing, is defined as a general term for unwanted (and, in general, unknown) modifications that a signal may suffer during capture, storage, transmission, processing, or conversion. When penetrating or intraneural electrodes are used to address this problem, nerve signal quality is improved at early time points, but the signal decays over time due to foreign body host response processes (inflammatory responses) and due to the micro-motion of the position of the tethered electrodes, in relation to surrounding soft tissues into which they are embedded. Additionally, there is the inherent problem that when an electrical stimulation is applied, there are large, sustained noise artifacts. Prior art attempts to address this have been through efforts at blanking out the noise, rather than subtracting out the noisy signals, thereby losing neural responses that may be measured locally with greater accuracy. Epineural electrodes, such as cuffs, provide a robust and stable interface for recording whole-nerve neural activity, but the electrical isolation of the epineurium/perineurium reduces the magnitude of signals, resulting in a lowered SNR. In particular, the biological noise in epineural electrodes can significantly distort recordings and even bury the neural signals, if the precision of the recorder is not sufficiently high. Post-signal processing to remove biological noise often creates new noise artifacts that can be confounded with nerve signals. Due to different chemical compounds, cell tissue environments, and tissue responses to implants, the electrode-tissue interface noise can be very different from a measurement in a bench testing apparatus. Several models and more recent measurement data suggest that a large portion of electrode noise is non-Ohmic (Johnson-Nyquist noise, or thermal noise), which is the electronic noise generated by the thermal agitation of the charge carriers (usually the electrons) inside an electrical conductor at equilibrium, which happens regardless of any applied voltage; for such noise, the sensing electronics can play a role in reducing the electrode noise. The amount of electronics noise in nerve recordings can be quite large due to elevated filter noise. For example, when recording a nerve in the abdomen, the constant motion of viscera causes large amplitude-motion artifacts. To avoid saturating electronics, an add-on analog filter with a corner frequency at least ten times higher than the artifact frequency is required. This ten-fold requirement increases the analog filter noise in the neural signal band up to tens of gV and beyond. The unmet need to improve neural recording signal requires a novel solution that goes beyond the optimization of individual noise components.

Inabilities to extract signals of individual fascicles and inabilities to develop chronic stability. Noninvasive nerve interface approaches provide small signals from a highly limited number of electrodes. Current technologies have demonstrated that recording compound nerve action potentials (CNAPs), mostly from exposed nerves, are sufficiently greater than the background noise activities, but CNAPs constitute such a volley of synchronous action potentials that it is not possible to deconvolute them into individual fiber activities, which is required to perform neural decoding. Spontaneous or less-synchronized neural activities from a nerve are much smaller than CNAPs, and particularly true of cuff electrodes, it is seen that such signals are further attenuated by the physical presence of the nerve sheath. For example, it has been confirmed in the prior art that after using neuromuscular blockers, surface canine vagus nerve signals are in the range of microvolts to tens of microvolts for de-sheathed nerves, but are even smaller (estimated at less than 10 μV) when the epineurium was intact during surface recordings. A nerve is an enclosed, cable-like bundle of many myelinated nerve fibers, structured as sheaths. The endoneurium is a layer of connective tissue around the myelin sheath of each fiber. The endoneuria with their enclosed nerve fibers are bundled into nerve fascicles, and each fascicle is within its own protective sheath called a perineurium. Multiple fascicles are bundled within yet another sheath, the epineurium. These protective sheaths isolate fibers, making it extremely hard to record and modulate signals on individual fibers from the outside of the nerve. Invasive intraneural electrodes may produce detectable signals at early time points, but signal decay over time due to tissue response has remained a problem. Initial insertion of the electrodes causes an early host immune system foreign body response leading to tissue encapsulation, with extensive tissue damage resulting from the micro-motion of the tethered electrodes relative to the soft tissue. Due to a lack of chronic stability, intraneural electrodes pose a significant long-term usage challenge. Therefore, there is still a need to pursue non-penetrating neural interfaces with significantly reduced noise to be able to accurately record small nerve signals.

Inability to record microvolt-level nerve activity, while in parallel, stimulation pulses are being applied to the same nerve. During the application of stimulation pulses, electrode-generated stimulation artifacts often produce a shift in the differential mode signal and the common mode signal, both of which need to be properly rejected. Subtraction of the stimulation artifact requires compensating for the frequency-dependent amplitude and phase of the transfer function. This subtraction process cannot be done as a matter of routine because the process requires a very high precision amplifier buffer to store the collection of superimposed artifacts and the collected neural signals. As noted, in brain recording experiments, the amplitude of neural signals (spikes and field potentials) is seen to be around a few hundred gV, but the amplitude of stimulation artifacts can reach as great as several Volts. To deal with a signal amplitude that can be less than 10 μV, there is a requirement for higher precision electronics capable of carrying out linear amplification. Every extra bit of increased precision of this type requires a 4-fold increase in the supplied power, which is consistent with the thermal slope in commercial analog electronics. As a result of this phenomenon, a commensurate upgrade of present-day brain technologies that could support simultaneous recording and stimulation on a peripheral nerve would require a multi thousand-fold increase in power.

Mismatch error. A central problem in the development of neurostimulator designs for electrical stimulation of neurons is referred to by those of ordinary skill in the art as that of mismatch error. In a signal processing system several operations or computations are performed on a signal in different stages sequentially. Each of these operations seeks to emphasize a wanted component or property of the signal of interest, without adding too much, or too many, unwanted extra components. These unwanted extra components are now known to be due to the non-idealities of the circuit implementation compared to the designer-specified operation of the circuit. Circuit non-idealities can be divided into two groups: random errors, and systematic errors.

In this field, random errors are the result of the stochastic nature of many physical processes. The stochastic behavior of charge carriers in a conductor, for instance, results in various types of noise signals and the stochastic nature of the physical phenomena that take place during the fabrication of integrated circuits, results in a random variation of the properties of the fabricated on-chip devices and mismatches between identically designed devices.

Systematic errors occur because a typical circuit implementation only approximates an ideal signal processing operation to a limited extent. These errors are caused, for instance, by the non-linear operating characteristics of devices or by the influence of parasitics (e.g. unwanted resistance, capacitance, or inductance in a circuit element) in the signal path or device structure.

The effect of these non-idealities can be of different kinds. The noise signals limit the minimal signal that can be processed with the system. Device mismatch limits the accuracy of the circuit behavior and again limits the minimal signal or energy that is required to execute meaningful signal operation functions. For linear systems, the non-linearities of devices generate distortion components of the signals or modulate unwanted ‘noise’ signals into the signal band that is being used. This typically limits the maximal signal that can be processed correctly.

The circuit designer of ordinary skill in the art has heretofore attempted to reduce the effect of such distortion non-idealities by using small modulation indices for the bias signals. By using large device sizes, the impact of mismatch is lowered and by using low impedance levels, the thermal noise signals are reduced. Such measures have, however, very important consequences on the power consumption and operational speed of the system. Therefore, the quality of a circuit realization is evaluated from the obtained accuracy, noise level, or linearity relative to the used power and the speed of operation, all in an effort to achieve, for a given speed, the best performance with a minimal power consumption.

Transistor mismatch. In the specialized case of transistor mismatch, the circuit designer of ordinary skill can reduce the effect of distortion non-idealities by the use of small modulation indices for the bias signals; so by using large device sizes, the impact of mismatch is lowered, and by using low impedance levels, the thermal noise signals are reduced. These measures have, however, very important consequences on the power consumption and operation speed of the system. Therefore, the quality of a circuit realization is evaluated from the obtained accuracy, noise level or linearity relative to the used power and the speed of operation. The designer will try to achieve—for a given speed—the best performance with a minimal power consumption. Prior art knowledge in the field of transistor mismatch held that such mismatch puts a fundamental limitation on the maximal total performance of analog signal processing systems. The Speed-Accuracy/Power ratio is fixed by technological constants that express the matching quality of the technology.

Inherent transistor mismatch. Any two identically-designed devices on an integrated circuit have random differences in their behavior and show a certain level of random mismatch in the parameters which model their behavior. This mismatch is due to the randomly-determined, or stochastic, nature of physical processes that are used to fabricate such devices. Mismatch is thus the process that causes time-independent random variations in physical quantities of identically designed devices. For example, the mismatch of two complementary symmetry metal-oxide silicon transistors (CMOS), that are identical in their manufacture, is characterized by the random variation of the difference in their threshold voltage VTO, their body factor γ and their current factor β. For technologies with a minimal device size larger than typically 2 μM, a widely accepted and experimentally verified model for these random variations is a normal distribution with mean equal to zero and a variance dependent on the gate-width W and gate-length L and the mutual distance D between the devices. The characterization of transistor mismatch is a tedious process which requires a very large measurement effort. The design, realization, and validation of the measurement set-up, the acquisition of the experimental data, and the statistical processing of the data have to be performed with great care to avoid errors and systematic effects. Moreover, when migrating towards sub-micron and deep-sub-micron technologies, the standard mismatch models have to be checked for their validity; if necessary, the effects of the short or narrow channel effects have to be accounted for in model extensions. A general relation can be derived for the minimal power consumption of a signal processing system due to the effect of the mismatch in the components. Transistor mismatch (as well as the phenomenon of thermal noise) are both random processes and put a limit on the smallest signal that can be processed in a circuit; both phenomena will impose a minimal power consumption to achieve a certain drive, specification, and speed.

Exemplary prior art efforts and attempts to control and reduce mismatch are discussed in “Implications of transistor mismatch on analog circuit design and system performance”, https://www.ee.columbia.edu/˜kinget/papers files/chapter3.pdf, the entire disclosure of which is incorporated herein by reference, and which readily shows the great degree of challenge and importance placed on the problem of reducing mismatch. The origins of transistor mismatch, on the other hand, are linked to the device structure, and device physics, and to the fabrication technology of integrated circuits. Device mismatch originates from the stochastic nature of physical processes used for the fabrication of devices, such as ion implantations, diffusions, or etching. The device structure using a channel in a doped material and its operation by modulating the channel resistance result in random fluctuations of the device's properties and operation. For integrated circuit technologies as we use them and fabricate them today, these physical limitations are very fundamental and device mismatch is unavoidable. From this perspective, the limits imposed by device mismatch are restricted to signal processing systems realized using integrated circuits. As such, they are of course very important in the quest for minimal power consumption in integrated circuits. Prior art attempts to cope with the above described challenges include the following fields.

### SUMMARY OF THE INVENTION

The present invention is a neuroprosthesis device, comprising a nerve interface, an artificial intelligence engine, an artificial intelligence neural decoder run by the artificial intelligence engine, and an electromechanical prosthetic limb. The nerve interface is itself comprised of a frequency shaping neural recorder and a redundant crossfire neural stimulator. The nerve interface is configured to establish bidirectional neural recording and neural stimulating communications with one or more selected residual peripheral nerves afferent from the brain, running through an arm or through a leg. The frequency-shaping neural recorder and the redundant crossfire neural stimulator are so configured as to establish the bidirectional recording and stimulating communications in near-simultaneous time, and in a most preferred embodiment of the present invention, in simultaneous communication activity. The artificial intelligence neural decoder is configured to execute a deep learning architecture, which gathers inputted nerve data from an amputee's movement intentions or motion intentions of a healthy limb, and translates such data into control of the electromechanical prosthetic limb that is taking the place of an amputated limb, which can be an arm, a hand, an upper leg, a lower leg, a foot or any anatomically correct combination of limbs. Preferably, the artificial intelligence neural decoder in integrated in a prosthetic arm & hand unit.

By virtue of the direct neurological control of the prosthetic device, in contrast to the prior art devices that rely on control of the prosthetic device by the actions of surviving muscles adjacent or in proximity to the terminus of the amputation, The device of the present invention is more correctly thought of as being a neuroprosthetic limb or hand. The actions by the user are still manifested through e.g. an electromechanical hand and digits, but the control of the device is through neurological signals from the brain, instead of through interpretation of muscular movements elsewhere on the limb. The device of the present invention translates signals from the brain into mechanical movement of e.g. wrist and digits so quickly, and so closely mimicking the movements of a natural hand, that the device effectively functions as a phantom hand that replaces an amputated hand. Such a neuroprosthetic phantom hand achieves its performance through ultra fast reception and translation of brain signals that are processed by the artificial intelligence engine. The artificial intelligence engine is configured so as to control the prosthetic wrist and its prosthetic fingers through user-controlled intuitive movements and motions characterizable as those of a natural wrist and natural fingers. The device most preferably has prosthetic fingers that additionally have mounted on them touch-sensitive sensors, and the sensors are configured to generate microstimulation patterns. These microstimulation patterns that are modulated by the artificial intelligence decoder and then to provide somatosensory feedback to the prosthetic wrist and fingers.

In its most preferred embodiment, the device is configured so as to sequentially collect training data from the user's brain and to use such training data to train the artificial intelligence neural decoder to in turn provide a trained model of movement, which is additionally trained through received motions of the prosthetic wrist and the prosthetic fingers, and thence to deploy the trained models to the future generation of motion through movements of the prosthetic wrist and the prosthetic fingers.

The nerve interface comprises an artificial intelligence engine, which is a fully integrated bioelectronics unit, and a number of a number of fascicular microelectrodes that are implanted into selected nerve fibers in selected nerves of an amputee's peripheral nervous system, thereby connecting those selected nerve fibers with the artificial intelligence engine and thereby constituting the source of inputted brain data into the engine. The engine further comprises a plurality of microelectronic microchips configured so as to establish neural recording and neural stimulation simultaneously, instead of waiting on the intake, recording, and processing of input data to then sequentially release a batch of output data. The microelectronic microchips in the engine comprise at least one frequency-shaping amplifier circuit configured to obtain ultra-low energy noise nerve signals, and to simultaneously suppress undesirable signal artifacts to present high signal to noise ratio inputs into the processing units of the engine. The artificial intelligence engine of the neural interface also comprises a high-precision analog to digital converter to present converted signals to the amplifier circuit and then on to the processing units. A small standalone computer means, e.g. a system-on-chip mini-computer module that is in the engine receives digitized and cleaned-up signal data, processes the data, and communicates the processed data outputs to a real-time motor signal decoder circuit. The preferred system-on-chip comprises an integrated central processing unit, a graphics processing unit, a random access memory, a flash storage means, and the system then is configured so as to deploy artificial intelligence software in an autonomous application fashion. The graphics processing unit itself is comprised of a number of computer unified device architecture parallel processes, which are configured so as to run a deep learning library. Examples of deep learning libraries include, but are not limited to, TensorFlow, PyTorch, Caffe, Caffe 2, Chainer, CNTK, DSSTNE, DyNet, Genism, Gluon, Keras, Mxnet, Paddle, or BigDL. TensorFlow, PyTorch, and Caffe are especially preferred. By careful selection of a graphics processing unit and coordinating it with a selected deep learning library, an engineered balance can be achieved between electrical power consumption and robustness of the selected deep learning library. The ideal such balance becomes important in order to find the smallest power source (which will usually comprise a rechargeable battery means) and the powered unit, to minimize bulk and weight of the prosthesis.

Where the prosthesis is a hand or hand/arm device, the hand will be under the control of a hand controller circuit downstream of the engine, which hand controller will in turn be comprised of a requisite number of microcontrollers to control the actions of the fingers and their individual phalanges and joints. The prosthetic hand will further be comprised of a power supply for the hand controller, a number of direct current motors for each digit of e.g. a hand, which will be individually actuated by electrical signals received from the microcontrollers, in response to decoded movement signals generated by the deep-learning predictions calculated by the artificial intelligence engine. Also, the device of the present invention will comprise an input/output unit configured to receive and transmit data during the operation of the neural interface, and appropriate memory storage capacity configured to store data requisite for the operational needs of the prosthetic device as a system.

### BRIEF DESCRIPTION OF THE DRAWINGS

FIG. 1 is an illustration of a system capable of interfacing with an arm nerve, capturing neurological signals therein, processing the signals, and converting the nerve signals into suitable signals to motors in a prosthetic hand, thus illustrating the basic circuit components of the invention.

FIG. 2 is a photographic image of an assembled prototype neuroprosthetic forearm and hand of the invention, illustrating externally visible components.

FIG. 3 is a set of three photographic image of an assembled prototype neuroprosthetic forearm and hand of the invention fitted to and being worn by a human subject.

FIG. 4 is a photographic image illustrating exemplary placement of microelectrodes used in the invention, in position relative to two major nerves of the human forearm.

FIG. 5 provides an illustration of the relative positioning of an implantable nerve interface used in the invention, in relation to major locations of the completed neuroprosthetic hand.

FIG. 6 is a photographic image illustrating the top and bottom aspects of a preferred embodiment of a nerve interface system sub-assembly of the neuroprosthetic hand.

FIG. 7 is a photographic image illustrating the top and bottom aspects of a preferred embodiment of an artificial intelligence electronic component sub-assembly of the neuroprosthetic hand.

FIG. 8 is an illustration of the dorsal aspect of the neuroprosthetic hand, illustrating the relative positioning of its power supply, micro driver, and microcontroller.

FIG. 9 is a photographic image of two alternative preferred embodiments of the neuroprosthetic hand of the invention and its forearm-positioned major sub-assemblies.

FIG. 10 block diagram of the flow of data through the artificial intelligence sub-assembly of the neuroprosthetic hand.

FIG. 11 is a table of mathematical functions variously applied in motor decoding of neurostimulatory signals from the brain to the arm and thence to the hand.

FIG. 12 is a simplified block diagram showing an exemplary flow of computational functions applied in the design of deep learning artificial intelligence neural decoding.

FIG. 13 is a photographic image showing a hand amputee in an experimental setup to collect a dataset based on signals generated by having the subject move their natural hand in prescribed motions to record brains signals as the natural hand is moved.

FIG. 14 is a simplified block diagram showing a procedure for artificial intelligence data generation without the use of a large mainframe-type computer.

FIG. 15 illustrates a set of graphs that plot the parameters of a stimulation pattern in real time for amplitude, pulse-width, frequency, or a combination of all three parameters as an analog curve of touch sensation data.

FIGS. 16A and 16B are graphical representations of the results of overall time latency through multiple embodiments of artificial intelligence data processing and of decoding rates as a function of varying power modes.

FIG. 17 is a graphical representation of prediction outcomes and probability computed over multiple validation datasets.

FIG. 18 is a tabular graphical representation of the classification performance results from an amputee's individual fingers.

FIG. 19 is a set of photographic images of an amputee testing their prosthesis in a laboratory setting, training a neural decoder model through a variety of hand movements.

FIG. 20 is a set of photographic images of an amputee testing their neuroprosthesis in a non-laboratory, real-world, environment.

FIGS. 21A, 21B, and 21C are a set of photographic and graphic images of representations of an amputee using their neuroprosthesis and how such motion generates touch data by amplitude as a function of time, and of how such activity improves tactile discrimination accuracy over multiple practice sessions.

FIG. 22 is a simplified diagram illustrating an alternative embodiment of the invention illustrating human control over mechanical devices in a telekinesis manner.

FIG. 23 is a photographic image of an amputee controlling action in a computer monitor to map their neurologic signals to finger movements in both a neuroprosthesis and a computer database.

FIG. 24 is a photographic image of the use of an implanted nerve interface to control action in a computer video game.

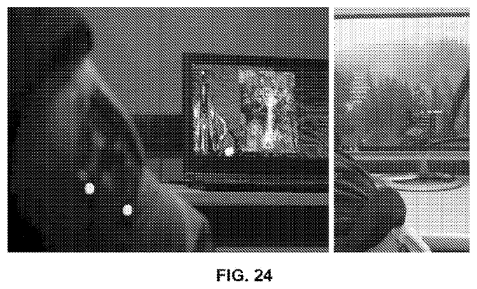

Sciences and technologies used in the application of the invention. Deep neural network to process nerve neural signals. The ultimate goal of an upper-limb neuroprosthesis is to achieve dexterous and intuitive control of individual fingers. Previous literature shows that deep learning (DL) is an effective tool to decode the motor intent from neural signals obtained from different parts of the nervous system. However, it still requires complicated deep neural networks that are inefficient and not feasible to work with in real-time. Different approaches were incorporated herein to enhance the efficiency of the DL-based motor decoding paradigm. First, a comprehensive collection of feature extraction techniques was applied to reduce the input data dimensionality. Next, two different strategies were used for deploying DL models: a one-step (1S) approach when big input data were available and a two-step (2S) when input data were limited. With the 1S approach, a single regression stage predicted the trajectories of all fingers. With the 2S approach, a classification stage identified the fingers in motion, followed by a regression stage that predicted those active digits' trajectories. The addition of feature extraction substantially lowered the motor decoder's complexity, making it feasible for translation to a real-time paradigm. The 1S approach using a recurrent neural network (RNN) generally gave better prediction results than all prior art machine learning (ML) algorithms, with mean squared error (MSE) ranges from 10⁻³ to 10⁻⁴ for all fingers while variance accounted for (VAF) scores are above 0.8 for the most degree of freedom (DOF). This result reaffirmed that DL is more advantageous than classic ML methods for handling a large dataset. However, when training on a smaller input data set as in the 2S approach. ML techniques offered a simpler implementation while ensuring comparably good decoding outcomes compared to the DL ones. In the classification step, either machine-learning (ML) or DL models achieve accuracy and an F1 score of 0.99. Thanks to the classification step, in the regression step, both types of models resulted in comparable MSE and VAF scores as those of the 1S approach. Recording nerve neural signals with cuff electrodes is an important milestone towards developing a high-performance, minimally invasive neural interface. The thrust of this is to develop tools to analyze and understand the observed neural signal recordings. Differing from a brain single-unit recording which contains activities of a few neurons, a cuff electrode records neural signals from a nerve bundle of thousands of axons, where the recorded signals can vary in shape and in pattern, and are characterized in having poorer signal to noise ratios. Therefore, methods commonly used to process brain signals (for example, spike sorting, firing interval engineering, and rate based codings) will be less effective in processing nerve neural data. The ability to separate weak neural signals from background noise is crucial in nerve signal processing. Signal detection is preferably accomplished in a preferred embodiment of the invention by using a modified deep variational autoencoder (VAE) means for signal detection. An exemplary deep VAE consists of sequentially connected encoder and decoder networks, where the encoder learns a class label y and a probability distribution of the code z with stochastic variables of the input data x, and the decoder aims to reconstruct the input based on the class label and the code. Use of such a deep VAE means may enable the development of a large-scale, well-annotated nerve dataset, as well as a thorough exploration of inputted signals and noise, and the generation of representations of the collected and inputted signals and noise, all through the use of the deep VAE, which in turn enables the enforcement use of a de-noising criterion such that the noise will be maximally removed. After such a training phase, the deep VAE will be able to de-noise the received data in a subject or patient and hence to improve signal detection. The present invention's novel method of use of a conventional VAE is performed in combination with a novel dataset and a novel de-noising algorithm.

Datasets. Deep learning algorithms rely on large-scale, well-annotated datasets to achieve a superior performance. For example, ImageNet, a large-scale visual database designed for use in visual object recognition software research, contains over 14 million hand-annotated images, and is considered by those of ordinary skill in the art as having enabled a revolution in deep learning. The database of annotations of third-party image URLs is freely available directly from ImageNet at https://www.image-net.org. In the present invention, to bridge deep learning and neural signal processing, a dataset similar to the taxonomy and annotation strategy of ImageNet is first constructed, according to procedures and processes well known to those of ordinary skill in the AI arts. The dataset then is used for developing neural signal processing algorithms.

Dataset generation. Cuff electrode data are a data summation from both filtered intraneural signals (signals within the nerve) and from noise arising out of external sources. Normally, cuff electrode data (both signals and labels) are not available for learning algorithms, especially for supervised learning, to record high quality, multi-site intraneural signals, and must be generated as part of the practice of the invention. We have now built a cuff electrode dataset based on intraneural signals. First, a finite element model of epineurium is developed to simulate cuff electrode neural signals based on multi-site intraneural signals. To this database is added noise that has been segmented from cuff electrode recordings, and the procedure is repeated with data from different electrodes and animal model preparations. This yields a dataset derived from real experiments that can support the development of supervised learning algorithms to process neural signals.

Deep learning de-noising. Mathematically, the data representation process with the deep VAE can be expressed as:

$$p θ ( x , y , z ) = p θ ( x \vert y , z ) p ( y ) p ( z )$$

where pθ(x|y, z) quantifies how the observed values of x are related to the latent random variables y and z, and p(y), p(z) represent a known prior distribution of the latent variables y and z. Given this representation model, the posterior distribution pθ(y|z, x) can be used to infer y, z and to find parameters θ that maximize the marginal likelihood pθ(x). To approximate the intractable pθ(y|z, x) a decoding distribution qΦ(y|z, x) is modeled by learning the parameters Φ from the data. Next, consider a 1-D time-series input xt={Xt−T1, . . . Xt+T2}, where X represents a single-electrode recording and [t−T1, t+T2] is a temporal scanning window. The binary classification label (i.e., neural signals or noise) at time t is denoted as a one-hot vector yt, and the corresponding latent variables are represented as zₜ. The preferred embodiment of the deep VAE models a joint distribution according to pθ(xt|yt,zt) factorized as pθ(xt,yt,zt)=pθ(xt|yt,zt)p(yt)p(zt). For the decoder model, use pθ(xt|zt,yt)=N(μθ(zt,yt), σ2θ(zt,yt)I); for the encoder, relying on the theory of variational inference to approximate the intractable posterior pθ((z|x,yt) with a tractable auxiliary distribution qΦ(zt|xt,yt)=N(μΦ(xt,yt), σ2Φ(xt)I). In the supervised case with an annotated dataset, the label yₜ is observed, allowing the parameters θ and Φ to be optimized by maximizing the extended variational lower bound:

$$log p θ ( xt \text{?} , yt ) \geq Eq Φ ( zt \vert xt , yt ) [ \text{} log p θ ( xt \vert yt , zt ) + log p θ ( yt ) + log p θ ( zt ) - log q Φ ( zt \vert xt , yt ) ] = LL ( xt , yt ) .$$

$$\text{?} \text{indicates text missing or illegible when filed}$$

For de-noising a nerve recording, the user injects the cuff electrode noise ε into xₜ to synthesize cuff recordings ˜xt=xt+ε, where the noise is picked from the cuff data.

Simultaneous recording and stimulation on a peripheral nerve. Proof of concept of electroceuticals requires integrating the stimulation function and performing personalized adaptive neural modulation therapies based on neural feedback. Another challenge that the invention had to overcome is that nerve neural signals are very weak, and thus they are vulnerable to the stimulation noise artifacts. To reduce such noise artifacts, a key feature of the novel invention is that of redundant crossfire (RXF) stimulator design based upon redundant sensing theory.

Redundant sensing. Redundancy is a fundamental characteristic of many biological processes such as those in the genetic, visual, muscular, and nervous systems; yet its underlying causative driving mechanism is not well understood. A complete discussion of the phenomenon of redundancy is set forth at A Bio-inspired Redundant Sensing Architecture, accessible at http://papers.nips.cc/paper/6564-a-bio-inspires-redundant-sensing-architecture.pdf, the entire disclosure of which is incorporated herein by reference. The present invention utilizes the redundancy from materials engineering to enhance the accuracy and precision aspects of the system, by focusing on the application of the phenomenon of redundancy to reduce stimulation signal residual charge, and thus reduce the effects of stimulation noise artifacts. In the invention's use of redundant sensing, each entry of information can be represented by a plurality of distinct configurations or microstates, and there is a distinct subset of such microstates that will allow linear representation of the entries of information, and such a set or subset will not be bounded by the classic Shannon limit (see C. E. Shannon, The Mathematical Theory of Communication, by Claude E. Shannon and Warren Weaver. University of Illinois Press, 1964) when processed according to the practice of one preferred embodiment of the present invention. The invention's identification of an optimized subset is an NP-incomplete problem, but it is possible to find a sub-optimal solution with sufficient efficiency to obtain a well-operating final complete embodiment of the invention. For example, in the case where a target stimulus signal is a 100 μA biphasic current, with a 6-bit resolution in amplitude, the anodic and cathodic branches will have up to a 3% mismatch depending on electrode conditions and clock jitter. Thus, the mismatch current is 100 μA×(3%+½⁶)=4.5 μA, which represents the cause of the resulting residual charge and stimulation noise artifacts. It can be seen from this example that a given amount of mismatch is stimulus dependent, time variant, and sensitive to electrode-electrolyte offset, thus posing a significant challenge to effective reduction of residual charge and stimulation noise artifacts, without having to resort to the prior art strategies of increasing the amount of power consumed or increasing the size of the neurostimulation device, both of which are product design strategies that ultimately produce a finished device of poor ergonomics and disappointing user satisfaction.

RXR stimulator. Based on the redundant sensing strategy, the present invention comprises as one element a reverse crossfire (RXF) stimulator, wherein the outputs of two or more independent stimulation channels with a current-digital-to-analog converter (IDAC) output driver will effectively form a redundant sensing structure. See U.S. patent application Ser. No. 15/876,030, U.S. Ser. No. 15/864,668, U.S. Ser. No. 17/066,456, and U.S. Ser. No. 17/849,534, the entire disclosure and teachings of which are respectively incorporated herein by reference. The sensed redundancy is exploited to fine tune and achieve precise matching between the anodic and cathodic stimulation currents, thus suppressing the residual charge and stimulation noise artifacts.

Inherent challenges. The prior art suffers from multiple challenges: an inability to harness the full range of movements potentially possible from currently available dexterous prosthetic systems, for example the DEKA Arm, the APL Arm, and the DLR Hand/Arm systems; the efficacy of deep learning comes at the cost of computational complexity; the inefficiency of conventional central processing units (CPU) that are found on most low-power platforms; and that prior art deep learning models must be trained and deployed using graphics processing units (GPU), which have hundreds to thousands of multi-threaded computing units specialized for parallelized floating-point matrix multiplication. As for the necessary supporting software, prior art edge computing devices are compact hardware and therefore attractive for use in prostheses, and are suitable for deep learning uses, but current software is limited to highly customized neural networks, which hinders full potential of, for example, a preferred embodiment of the invention as claimed, namely a neural decoder implementation based on recurrent neural network (RNN) architecture.

Prior art approaches do not adequately address the challenge of efficiently deploying deep learning neural decoders on a portable, edge computing platform, and translating existing benchtop motor decoding experiments into real-life applications toward long-term clinical uses. Many studies have demonstrated the superior efficacy of deep learning approaches compared to conventional algorithms for decoding human motor intent from neural data. However, the application of deep learning on portable devices for long-term clinical uses has remained challenged due to the high cost of computational complexity. It is well-known that running deep learning models on conventional the type of CPUs found on most low-power platforms is hugely inefficient. The vast majority of deep learning models in the prior art must be trained and deployed using GPU, which has hundreds to thousands of multi-threaded computing units specialized for parallelized floating-point matrix multiplication.

Innovative Elements. The invention herein disclosed and claimed efficiently implements deep learning neural decoders in a sufficiently portable platform for clinical neuroprosthetic applications, made feasible by combining and integrating multiple innovative elements that we have chosen to combine and apply across various of the system's components. A first innovative element lies in using the development of an intrafascicular microelectrode array that connects nerve fibers and bioelectronics, as presented in Overstreet et al. Fascicle Specific Targeting For Selective Peripheral Nerve Stimulation. Journal of Neural Engineering, 16(6), 066040. (2019), the disclosure of which is incorporated herein by reference. The second innovation lies in the novel incorporation of a design of Neuronix® neural interface microchips, that allow simultaneous neural recording and stimulation, as presented in Nguyen & Xu et al. A Bioelectric Neural Interface Towards Intuitive Prosthetic Control For Amputees. Journal of Neural Engineering, 17(6), 066001, describing our Scorpius device therein, and which is further described below, and Nguyen et al. Redundant Crossfire: A Technique to Achieve Super-Resolution in Neurostimulator Design by Exploiting Transistor Mismatch. IEEE Journal of Solid-State Circuits. (2021), the two disclosures of which are incorporated herein, describing redundant crossfire neural stimulator and somatosensory experiments. The third innovation lies in incorporating optimization of the deep learning motor decoding paradigm that results in significantly reducing the decoder's computational complexity, as described in Luu et al Achieving Super-Resolution with Redundant Sensing. IEEE Transactions on Biomedical Engineering, 66(8), 2200-2209. (2019), the disclosure of which is incorporated herein by reference. There, the aim was to achieve engineering information redundancy, built into the system's architecture, in order to exploit the phenomenon of random transistor mismatch, and to thereby enhance the overall effective resolution capability of the device. The application of RXF in the present invention involves combining the RXF (i.e., crossfiring) outputs of two or more current drivers in order to form a redundant structure. When properly configured, this novel redundant structure may produce accurate current pulses with an effective super-resolution that is beyond the limitation commonly permitted by the physical constraints. The fourth innovation lies in the implementation of software and hardware based on a state-of-the-art edge computing platform that could support real-time motor decoding, as is further described below.

Accordingly, a need exists for a solution to address the multiple challenges and shortcomings currently existing in the prosthesis industry. The present invention addresses the challenges of the shortcomings of the prior art in addressing the problems described above, in an integrated multi-prong approach by the features of: having a nerve interface comprising a frequency shaping (FS) neural recorder and a redundant crossfire (RXF) neural stimulator on a chip to establish near-simultaneous bidirectional recording and stimulating communications with residual peripheral nerves of interest; using an artificial intelligence (AI) neural decoder, based on a deep learning architecture, to translate, in real time, an amputee's movement or motion intentions, as gathered from the amputee's nerve data; having a portable, self-contained, battery-powered AI engine to run the AI neural decoder, where the decoder is integrated into an electromechanical prosthetic hand; having the capacity to be able to intuitively control individual finger and/or wrist movements just like a natural hand, manifested as the ability to control the electromechanical prosthesis by directly moving the prosthetic fingers and/or wrist so that the prosthetic hand effectively becomes a phantom hand, thereby elevating the electromechanical prosthesis to the status of a neuroprosthesis; being able to provide somatosensory feedback in real-time by modulating a microstimulation pattern generated by the neuroprosthetic hand having touch-sensitive sensors; and by applying a procedure to collect training data, train the AI neural decoder, and deploy the trained model on the prosthetic hand.

### DETAILED DESCRIPTION OF THE DRAWINGS AND OF THE INVENTION

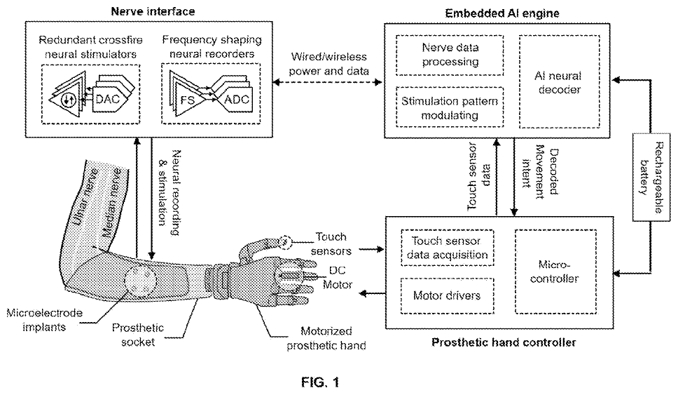

Turning first to FIG. 1, there is shown an overview of a preferred embodiment of the conceived neuroprosthetic system consisting of several components that actuate the prosthesis itself 130 having an upper arm 136 and a lower arm 138, namely a nerve interface 100 in contact with selected nerve fibers by microelectrode implants 140 that transmits and receives neural recording & stimulation signals 150, the nerve interface 100 comprising one or more redundant crossfire neural stimulator(s) 102, one or more frequency shaping neural recorders 104, connectably in communication with, via wired or wireless power and data signal stream 106, an artificial intelligence (AI) engine 108, comprising an artificial intelligence (AI) nerve data processing module 114, an AI neural decoder 110, and a stimulation pattern modulating module 112, a rechargeable battery means 116, a prosthetic hand controller module 122, comprising a number of microcontroller(s) 124, one or more motor drivers 126, and a touch sensor data acquisition module 128, a motorized prosthetic hand 144 in movable relationship via prosthetic socket 142 to lower arm 138, said hand 144 having a number of touch sensors 148 mounted at the tips of the prosthetic's fingers and in the palm of the prosthetic's hand 144, DC motors 146 installed in the fingers of the prosthetic hand 144 and in its wrist, and a mechanical hand itself 144 with motorized fingers 147, touch sensors 148, and controller 122. The nerve interface 100 consists of multiple Neuronix® chips, which have been disclosed in Z. Yang et al., “System and Method for Simultaneous Stimulation and Recording Using System-on-Chip (SoC) Architecture”, U.S. patent application Ser. No. 15/876,030, 2018., Z. Yang et al., “System and Method for Charge-Balancing Neurostimulator With Neural Recording”, U.S. Pat. No. 10,716,941, 2020; and Nguyen et al. “Redundant Crossfire: A Technique to Achieve Super-Resolution in Neurostimulator Design by Exploiting Transistor Mismatch, IEEE Journal of Solid-State Circuits, 2021 DOI: 10.1109/JSSC.2021.3057041 (2021) the entire disclosures of which are incorporated herein by reference. They are fully integrated, multi-channel application-specific integrated circuits (ASIC) that can support high-resolution neural recording and stimulation. The neural recorders are designed based on frequency shaping (FS) architecture and high-precision analog-to-digital converters (ADC). They are capable of obtaining ultra-low noise nerve signals while suppressing undesirable artifacts. The neural stimulators are designed based on our redundant crossfire (RXF) architecture described in Nguyen et al, supra. The neural stimulators can then deliver a precisely controlled amount of charge to modulate neural circuits' behavior.

The neural recorders and stimulators of the invention interface with an amputee's peripheral nerve through multiple microelectrode arrays 140 that are surgically implanted into the individual's nerve fascicles in the forearm. Most preferably, the ulnar 132 and median 134 nerves are used, since they control the movements of the fingers and wrist, and since they carry the hand's somatosensory perception neurosignals (e.g., touch and proprioception). Other preferred embodiments may also include the radial nerve, which controls certain wrist movements and which carries additional somatosensory perception. Raw nerve data acquired by the neural recorders 110 are directly streamed to the AI engine nerve data processing circuit module 114 for further processing via a wired or a wireless connection 106.

The AI engine 108 is powered by a miniaturized, low-power edge computing device. The edge computing device is essentially a mini-computer equipped with dedicated hardware such as a central processing unit (CPU) and graphical processing unit (GPU) to perform data processing and deep learning inference. Here, fully-trained AI neural decoders 110 based on deep learning architecture are deployed to translate nerve signals of the amputee's true intentions to individual finger movements in real-time in the form of predictions made by the AI architecture, based on the input of the amputee's true intention neurological signals. The final predictions are sent over to the hand controller 122 to actuate the prosthetic hand 144. The AI engine also uses data from the neuroprosthetic hand's fingertips touch sensors 148 as additional data from the touch data acquisition circuit 128 to modulate stimulation patterns to create somatosensory feedback.

The mechanical hand can be modified from any existing commercial system with individually motorized fingers and/or wrist. Off-the-shelf touch sensors 148 are attached to the fingertips and palm to sense the force generated when the hand grasps an object. Another component of the invention is a customized microcontroller(s) 124 that receives decoded movement intents/intentions 118 from the AI engine and independently drives the finger and thumb motors accordingly. This controller also acquires touch sensor data 120 and relays such data to the AI engine 108. A rechargeable Li-ion battery 116 powers the entire system. Additional voltage regulator circuits are included to generate the proper power supply for each component.

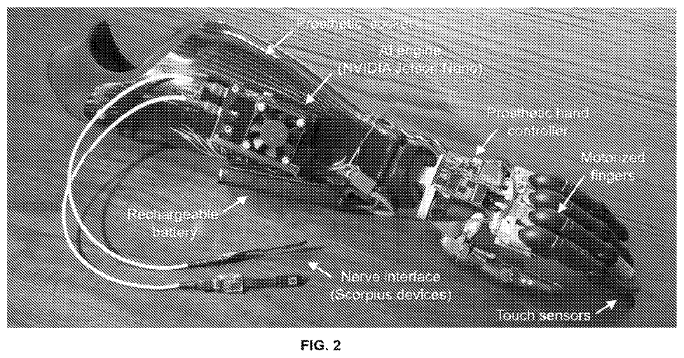

At FIG. 2 there is shown an actual functional prototype of a highly preferred embodiment of the prosthetic hand with embedded AI and neuro-feedback. The nerve interface includes two Scorpius® devices 100, although alternative embodiments of the invention can use other numbers and other members of the Scorpius® family of devices 100; each has two Neuronix® chips, to enable separate but simultaneous neural recording and stimulation. The Scorpius® device 210 communicates with the AI engine via a wired USB connection 101.

The AI engine module 108 in this example of a preferred embodiment of the invention is powered by the NVIDIA® Jetson Nano® computer module 202 (NVIDIA, California, USA). This AI engine module 108 is preferably equipped with the Tegra X1® system-on-chip (SoC) that has a Quad-Core ARM Cortex-A57® CPU and a 128-core NVIDIA® Maxwell-type microarchitecture GPU. This exemplary GPU has 472 gigaflops (GFLOP)s of computational power available for deep learning inference. The module can operate in a preferred 10 W power mode (4-core CPU 1900 MHz, GPU 1000 GHz) or a preferred 5 W power mode (2-core CPU 918 MHz, GPU 640 MHz). The prosthetic motorized hand 144, drawing its power from the rechargeable battery means 116, is based on the i-Limb® platform (TouchBionics®, Ossur, Iceland) which features five individually actuated motorized fingers 147. In a preferred embodiment of the invention, the i-Limb default driver is replaced, by procedures well known to those of ordinary skill in the art, with the inventor's customized hand controller 204, described below, which directly operates the DC motors 146 hidden and not here visible, in each finger. The controller 122 is designed around the ESP32 module (Espressif Systems, Shanghai, China) with control signals being distributed to one or more low-power microcontroller(s) 124. The touch sensors 148 fixably mounted at each fingertip and the prosthetic hand's palm may be resistive force sensors (FSR Series, Interlink Electronics, CA, USA) and/or capacitive force sensors (SingleTact®, Medical Tactile Inc., CA, USA).

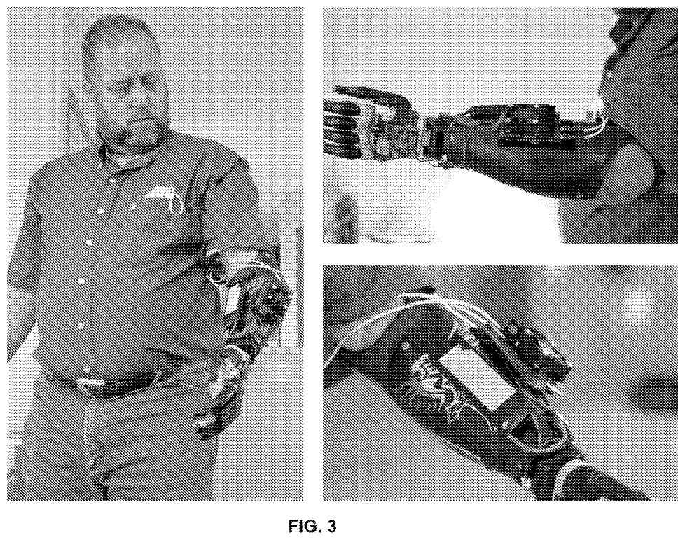

At FIG. 3 there is shown shows an amputee patient 300 displaying and wearing a highly preferred embodiment of a neuroprosthetic forearm 138 and hand 144 embodiment of the invention, as well as an dorsal view 302 and a. ventral view 304. For demonstration purposes, the nerve interface 100, AI-engine 108, battery 116, and prosthetic hand 144 were attached to the exterior of the amputee's 300 personalized prosthetic socket 142. In other alternative preferred embodiments, these components can be integrated into the interior of the socket 142, replacing the existing original equipment commercial EMG sensors and electronics.

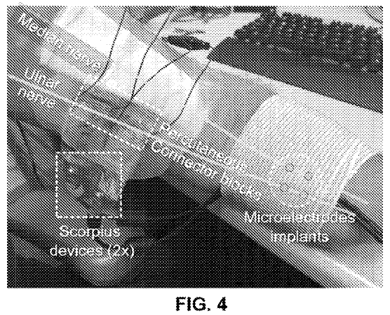

FIG. 4 shows the injured limb for the amputee patient 300 at greater magnification in ventral view, displaying the median nerve 400, microelectric implants 402, percutaneous connector blocks 404, Scorpius®-type devices 406, and ulnar neve 408. The amputee patient 300 had undergone an implant surgical procedure where four longitudinal intrafascicular electrodes (LIFE) arrays 402 were inserted into the residual median 400 and ulnar 408 nerves using a microsurgical fascicular targeting (FAST) technique, well known to hand and arm surgeons. Within one nerve, here the ulnar nerve 408, the arrays were placed into two discrete nerve fascicle bundles. The approximate location of the implanted hardware near the end of the stump was marked, with the location having been strategically located as far away as possible from residual muscles in the forearm in order to minimize volume-conducted EMG, a type of environmentally produced signal noise. The electrode wiring was brought out through percutaneous holes on the arm and secured into two connector blocks 404. The Scorpius® devices 100 were then attached to the percutaneous connector blocks 404 via two standard 40-pin Omnetics® nano-connectors.

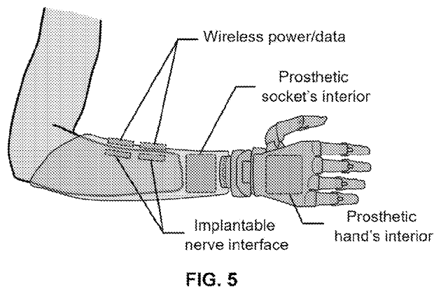

FIG. 5 illustrates a conceptual drawing of an improved alternative preferred embodiment of the neuroprosthetic arm and hand, showing wireless power and data modules 504, the boundaries of a prosthetic socket interior space 502, the boundaries of the prosthetic hand's interior space 504, and an implantable nerve interface 506. The implantable nerve interface 506 containing the Neuronix® chips is integrated with an array of microelectrodes 140 to create a fully implantable nerve interface device 506. The purpose of this is to eliminate the percutaneous wires present in alternative embodiments of the invention in which the nerve interface device is situated on the exterior of the skin of the patient, which makes the system suitable for long-termer usage. Two or more nerve interface implants 506 communicate with the AI engine 108 via wireless power 500, i.e. battery-powered, and wireless data telemetry. In addition, the AI engine 108 and battery 116 may be integrated into the prosthetic socket's interior space, while the prosthetic hand's interior space 504 may contain the controller sub-assembly and one or more additional batteries.

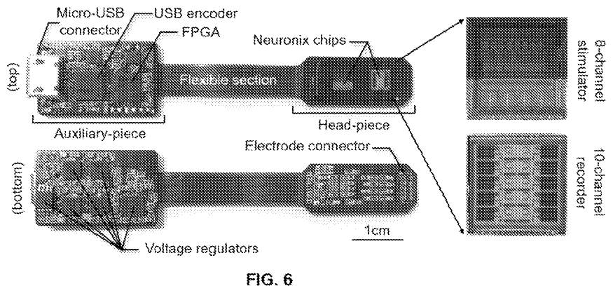

FIG. 6 is a photograph that shows the detailed layout of preferred embodiment of a Scorpius®-type nerve interface system in top view 600 and bottom view 601, with a micro-USB connector 602, a USB encoder 604, a field programmable field array (FPGA) 606, a flexible array section of interconnecting wires 608, two Neuronix® chips 610 and 611, a head piece 612, an enlarged view of an 8-channel stimulator 614, an enlarged view of a 10-channel recorder, electrode connector 618 and voltage regulators 620. The illustrated device is equipped with fully integrated Neuronix® chips to facilitate bidirectional simultaneous communications with the user's brain and nervous system via simultaneous electrical neural recording and stimulation, which is explained and described in U.S. patent application Ser. No. 15/876,030, 2018, the entire disclosure and teaching of which is incorporated herein. The illustrated device of FIG. 6 contains two Neuronix® chips, one being a 10-channel frequency-shaping (FS) neural recorder chip 616, likewise explained and described in patent application Ser. No. 15/876,030, and the other being an 8-channel redundant crossfire (RXF) stimulator chip 614, which is explained and described in U.S. patent application Ser. No. 17/849,534, the entire disclosures and teachings of which are incorporated herein by reference, to enable ultra-low noise level simultaneous neural recording and high-precision stimulation. In practice, multiple Scorpius®-type devices may be deployed depending on the number of channels required, it being understood that any of them could facilitate simultaneous recording and stimulation. The device consists of two sub-units, namely the head-piece 612 and the auxiliary-piece 613, connected by a flexible section of interconnecting wire array means. The head-piece 612 contains the Neuronix® chips 614 and 616, along with an electrode connector 618 and other passive components. The head-piece 612 is designed to physically separate the Neuronix® chips 614 and 616 from other off-the-shelf components to minimize avoidable noise coupling and improve signal to noise ratios. The auxiliary-piece 613 is comprised of an FPGA 606 (AGLN250, Microsemi, CA, USA), a USB interface 603 (FTDI, UK), and power management circuitry with various voltage regulators 620, for example here an ADR440, an ADP222, and an ADA4898-2, Analog Devices®, Massachusetts, USA. The auxiliary-piece's function is to pass through the digitized neural data and control commands from the headpiece Neuronix® chips, while powering the Neuronix® chips through a single micro-USB connector.

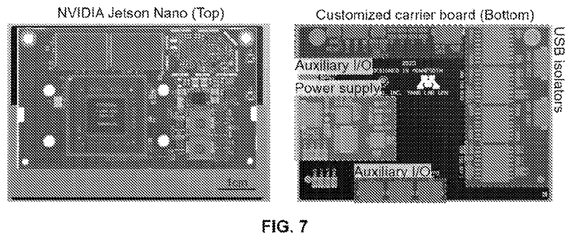

Turning now to FIG. 7, there is shown in top view 700 a preferred embodiment of an AI engine 108, which includes an NVIDIA® Jetson Nano® (Nano) module 700 and 702 in bottom view) and a customized carrier board 704 located directly under the module 700, configured to have similar length and width physical dimensions to the greatest extent possible, and USB isolators 706, an auxiliary I/O circuit 708, and a power supply management circuit 710. A 260-pin SODIMM connector, not shown, provides a sandwiched connective interface between the two boards. The power supply circuits 710 provides the main rail (5V, 2 A) for the Nano module and other voltages (e.g. 3.3V, 1.8V) used in the sub-assembly. USB isolators 706 based on ADuM5000 and ADuM4160 dc converters (Analog Devices, Massachusetts, USA) were used to reduce the deleterious impact of digital noise from affecting the Scorpius® neural recording system analog front-end performance. Other general-purpose I/O means 708 such as UART, I2C, USB (nonisolated), and the like, were also provided on the carrier board 704.

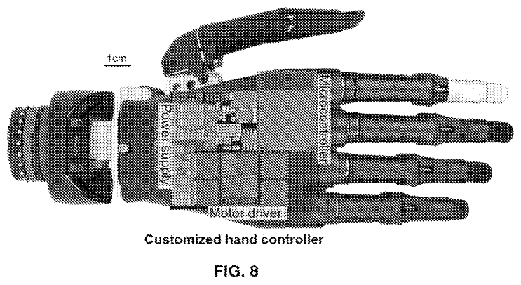

FIG. 8 shows a top (dorsal) view 800 of a customized hand controller board 801, which fits within the interior of the i-Limb® neuroprosthesis hand 144, and having a hand controller board 801, a microcontroller 802, a motor driver 804, and a power supply 806. In a preferred embodiment of the customized hand controller board 801, the power supply circuits 804 provide the (3.3V) rail for the ESP32 microcontroller module(s) 802 and for the (12V) rail for a motor driver circuit means 804. The motor driver circuit(s) 804 convert the ESP32 microcontroller 802 outputs into the pulse-width modulation (PWM) signals needed to actuate the DC motors within the hand (not shown in this figure).

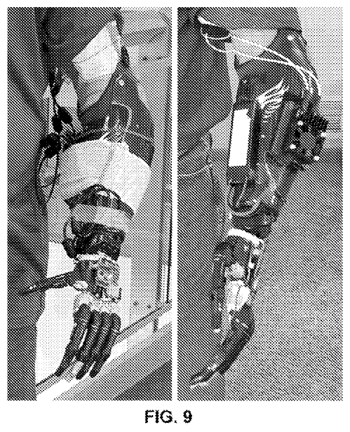

FIG. 9 shows two dorsoventral views 900, 902 of a fully assembled system attached to the amputee's existing socket, including a first preferred embodiment (left) 900 and a second preferred embodiment (right) 902. The first embodiment 900 uses the default Jetson® Nano's carrier board with off-the-shelf USB isolators and a power bank. While being seemingly bulkier, the first embodiment's motor decoding functions are identical to the second embodiment that features a customized printed circuit board (PCB) to reduce bulk volume.

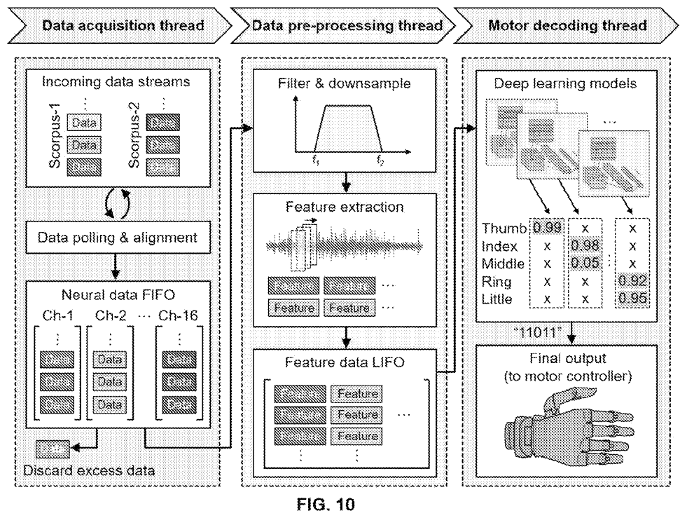

FIG. 10 shows an overview of the flow of the data processing steps deployed on the Jetson® Nano used in the AI subassembly of the invention. A program is implemented in a most preferred computer language, for example Python, and produces three separate threads—one for data acquisition 1000, one for data pre-processing 1010, and one for motor decoding 1018. This example of a most preferred multi-threading strategy implementation helps to maximize the utilization performance of the quad-core CPU used here, and reduces the processing latency.

The data acquisition thread 1000 polls data from the incoming data streams 1002 of two or more Scorpius devices and aligns them 1004 into appropriate channels. The data streams, one for every Scorpius device, continuously fill up the USB buffers at a bitrate of 1.28 Mbps per device. Each of the data streams 1002 contains data from eight channels, at a sampling rate of 10 kHz. Headers were also added to properly separate and align the data bytes into individual channels, and the data bytes were then placed into first-in-first-out (FIFO) queues 1006 and discarded excess data 1008 in preparation the data pre-processing thread 1010.

The data pre-processing thread 1010 filters and downsamples raw nerve data 1012, and subsequently performs feature extraction 1014 according to the procedures outlined in Luu & Nguyen et al., “Deep Learning-Based Approaches for Decoding Motor Intent from Peripheral Nerve Signals”, https://www.biorxiv.org/content/10.1101/2021.02.183.431483v1 (2021) the entire disclosure and teaching of which is incorporated herein by reference. This preferred embodiment data stream utilized nerve data in the 25-600 Hz band, which are known in the art to contain the majority of a neurosignals' power. We applied an anti-aliasing filter at 80% of the Nyquist frequency, downsampling by 2-times, and then applied the main 4th-order bandpass filter with 25-600 Hz cut-offs. In the feature extraction task 1014, each feature data point was computed over a sliding window of 100 msec with 20 msec increments, resulting in an effective feature data rate of 50 Hz. Feature is used here in the machine learning and statistics sense, meaning variable or predictor, so that a subset of the most relevant of such features can be assembled via various feature selection techniques to construct a behavior or activity model. Redundant or irrelevant features are removed by such techniques without much loss of information in order to shorten training times, simplify models, avoid dimensionality, and other uses. Here, the feature data were placed into last-in-first-out LIFO queues 1016 (rolling matrices) for the motor decoding thread 1018. Unlike prior art approaches, no data were stored for offline analysis. Nerve data were processed and fed to the motor decoder thread 1018 as soon as they were acquired or discarded if the thread cannot keep up. This LIFO setup ensures that the motor decoder thread 1018 always receives the latest, or most recently generated, data. In practice, the buffer to Python queue time is negligible, and the pre-processing time is the bulk of the non-motor decoding latency. Excess data could also be caused by small mismatches in clock frequency between different Scorpius® devices (a problem inherent in the device manufacturing process—see the discussion of transistor mismatch, above—which creates data streams with a higher bitrate than others. As a result, up to 60 msec of raw data is occasionally discarded.

Deep learning inference is the use of a fully trained deep neural network to make inferences or predictions on of from new or novel data that the model has never seen before. Here, the motor decoding thread 1018 ran deep learning inference by using deep learning models 1020 processing the most up-to-date feature data from the LIFO queues corresponding to the past 1 sec of nerve signals. For the neuroprosthetic hand, there were one to five deep learning models 1020; so that each model decoded the movements of one or more fingers. All deep learning models 1020 have the same architecture but may be trained on different datasets to optimize the performance of a specific finger. The reason for this is that while an individual deep learning model can produce a [5×1] prediction matrix, it is often difficult to train a single deep learning model that is optimized for all five fingers. For example, the first model in FIG. 10 only decoded the thumb movement. Because the control signals associated with the thumb are the strongest among the fingers for this particular example of an amputee, the thumb training typically converged in 1-2 epochs (the number of passes of the entire training dataset the machine learning algorithm has completed). Additional training could cause over-fitting (a model that has learned the noise instead of the signal is considered “overfit” because it fits the training dataset but has poor fit with new datasets). The final digitized prediction output 1022 is sent to the hand controller 1024 via a serial link for operating the robotic hand and/or to a remote computer via a Bluetooth connection for debugging Here the use of the term “remote computer” means that such digitized prediction outputs can be used not only for a neuroprosthetic limb, but also for an external robot of any sort, or for an electromechanical device of any sort, including video games, electromechanical toys, controllers of complex machines, and the like that have in common being under the partial or complete control of a computer.

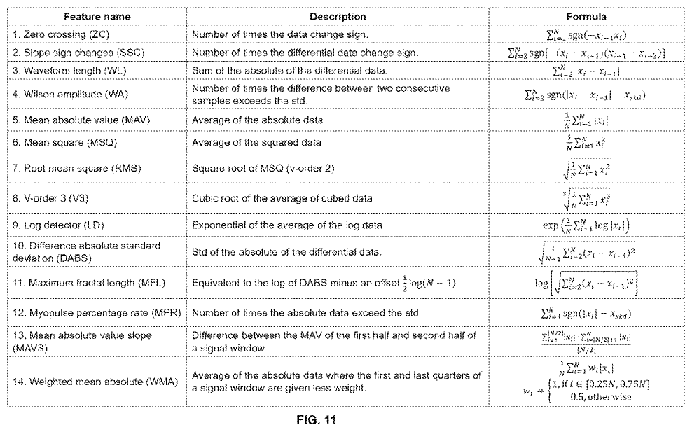

FIG. 11 shows the list, descriptions, and mathematical formulae for the assembly of the fourteen most relevant features, or variables and predictors, used for motor decoding. Listed here, the features are zero crossing, slope sign changes, waveform length, Wilson amplitude, mean absolute value, mean square, root mean square, V-order, log detector, difference absolute standard deviation, maximum fractal length, myopulse percentage rate, mean absolute value slope, and weighted mean absolute.

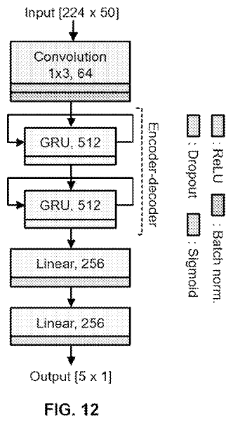

FIG. 12 shows a preferred embodiment design of a preferred deep learning AI neural decoder based on a recurrent neural network (RNN) architecture and implemented using the PyTorch library. The strategy of the AI neural decoder design was to reduce the input dimensions into the associated matrix down to five output matrix dimensions. In this preferred embodiment example, the input matrix dimensions were [224×50]=[16 channels, i.e. 8 per Scorpius® device in a two-device embodiment]×[14 features]×[50 time-steps]. Here 50 points of feature data at the effective 50 Hz rate corresponded to 1 sec of past neural data. The output matrix dimensions were [5×1] corresponding to the five fingers of a hand.

The input 1200 is fed into the initial convolutional layer 1201, which performs the convolution function 1203, and identifies different representations of data input. The subsequent encoder-decoder 1204 utilizes first 1026 and second 1208 gated recurrent units (GRU) to represent the time-dependent aspect of motor decoding. The two linear layers perform analysis on the decoder's output and produce the final output matrix 1210, which are the probabilities that an individual finger is active. 50% dropout layers are added to avoid over-fitting and improve the network's efficiency. Overall, each model consists of 1.6 million parameters in total.

The models were trained on a desktop PC with an Intel® Core i7-8086K, and an NVIDIA® RTX 2080 Super graphics card. We used the Adam optimizer method with the default parameters being β_{!}=0.99, β_{″}=0.999, and weight decay regularization L_{″}=10^{#S}. The mini-batch size was set to 64. The number of epoch (2-10) and initial learning rate (10^{#%}-10^{#&}) were adjusted for each model to optimize the performance while preventing over-fitting. The learning rate was reduced by a factor of 10 when the training loss stopped improving for two consecutive epochs. The training time for each epoch depended on the dataset's size and typically took about 10-15 msec.

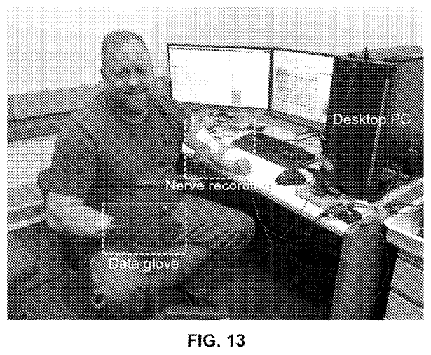

In FIG. 13 there is photographically shown an experimental setup utilized to collect a dataset with ground-truth labels to train the AI neural decoder used in this embodiment of the invention. The dataset was acquired with a desktop PC using the mirrored bilateral paradigm. Nerve signals were obtained from the arm of the amputated hand with the Scorpius® system, while labeled ground-truth movements were captured from the same amputee's able hand that is used a data glove 1300, in communication with a nerve recording set-up 1302, then on to a conventional desktop personal computer 1304. In each experiment session, the amputee was instructed to flex an able hand gesture 10 times, where the fingers are held in the flexing position for about 2 see. The data glove measured the angle of the finger's proximal phalanx with respect to its metacarpal bone. The data were then thresholded to produce the ground-truth labels for classification. For able participants, the training only included the flexing of individual fingers to verify the decoder's functions. For the amputee, we added different gestures where two or more fingers were engaged, such as fist/grip (11111), index pinch (11000), pointing (10111), and Hook 'em Horns (10110).

There were collected at least four or more mirrored bilateral training sessions for each hand gesture. Within a session, the patient performed a given gesture at different shoulder, arm, and body postures, recreating real-life conditions. Additional sessions may alternatively be required for gestures that are difficult to predict. The last data session, which contained the most up-to-date nerve data, was strongly preferred to be used for validation while the remaining were used for training. This configuration translates to a training-to-validation ratio two ranges of approximately 75:25 for able participants to 85:15 for an amputee.

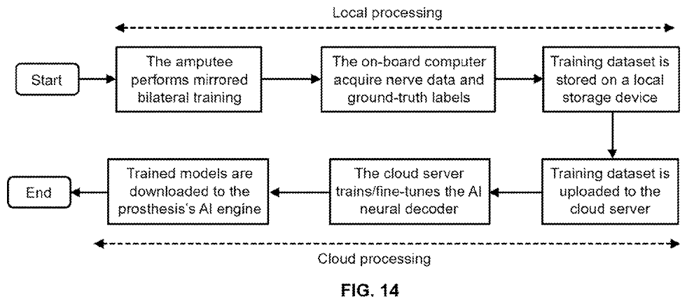

In the flow chart of FIG. 14, there is illustrated a preferred procedure to conduct an AI training & practice that does not require a powerful, specialized computer. The procedure can be done by local processing 1400 in a clinic or by an amputee themselves in a home. An amputee will perform mirrored bilateral training 1404 with a data glove 1300, following pre-defined instructions as described above. The prosthetic hand's on-board computer (e.g., the Jetson® Nano module) will directly acquire nerve data and ground-truth labels 1406 to create a resultant training dataset. This dataset is temporarily stored 1408 on a local storage device such as a flash memory device (e.g., a micro-SD card) or other storage device means. When the prosthetic hand is not being used, for example while its batteries are charging overnight, the on-board computer will upload the training dataset to a cloud server 1410. The cloud server processes the training dataset 1402 and then transmits to download to, and train or fine-tune, the amputee's AI neural decoder 1412, which would be personalized for that individual amputee. Once a course of training is complete, subsequent newly-optimized models are downloaded in the future to the neuroprosthesis's AI engine 1414 via an over-the-air software update. In practice, this procedure may be done every few months for adapting the decoder to compensate for any drift of the nerve interface over time.

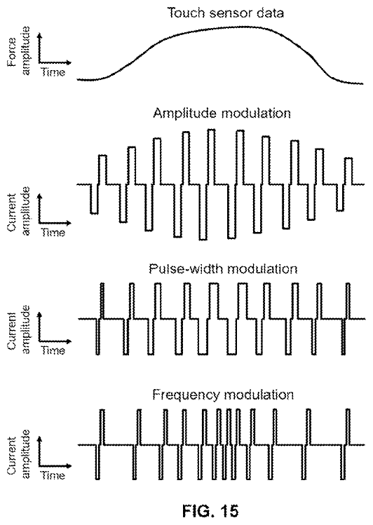

At FIG. 15 there are illustrated four modalities representing useful parameters of stimulation patterns in real time. These patterns are generated from touch sensor data 1400 from the fingertips of the neuroprosthesis and are used to subsequently capture somatosensation feedback that is useful in adjusting or fine-tuning the modalities of amplitude 1502, pulse-width 1504, frequency 1506, or a combination of all three modalities, the four of which will translate into improved-dexterity finger motions.

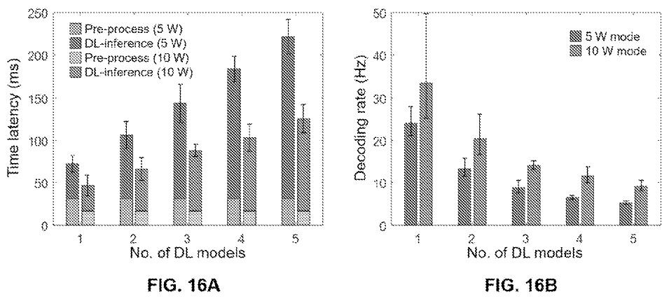

Performance results are shown at FIGS. 16A and 16B. FIG. 16A shows the overall time latency calculated from data acquisition to final classification prediction (i.e., time latency equals input lag). The metric of overall time latency is evidence of the system's responsiveness and is essential for optimizing real-time operation. The time latency mainly is comprised of the data pre-processing step and the deep learning-inference step. Only the time latency of the deep learning-inference step shows linear increases as additional deep learning models are used. The overall processing time is imposed by the CPU and GPU's clock speed which is subsequently limited by the power budget. It can be seen that switching the Jetson® Nano's power mode from 5 W to 10 W cuts down the latency approximately by half.

FIG. 16B shows the maximum throughput of the motor decoding pipeline, which measures the number of predictions produced per second (i.e., the frame rate). This metric also contributes to the system's responsiveness. We achieve a decoding rate that is unexpectedly significantly higher than the inverse of the time latency thanks to the multi-threading implementation described above. Like the latency, the decoding rate is also decided by the number of deep learning models and the Jetson® Nano's power budget, and it is seen that an increase in power both decreases time latency and increases decoding rate.

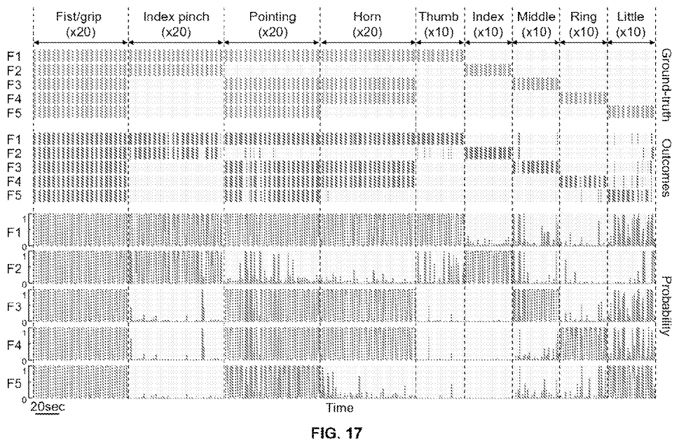

FIG. 17 is a graphical representation of the classification results, including the prediction outcomes and probability computed over the validation datasets. The size of the training/validation sets are approximately 30,000:10,000 for able participants and 150,000:26,000 for amputees, respectively. We use one model to predict all five fingers. The prediction results demonstrate that deep learning neural decoders could accurately predict individual fingers' movement, forming distinct hand gestures in the time series. The classification task's quantitative performance were evaluated using standard metrics, including the true positive rate (TPR) or sensitivity, true negative rate (TNR) or specificity, accuracy, and area under the curve (AUC), derived from true-positive (TP), true-negative (TN), false-positive (FP), and false-negative (FN).

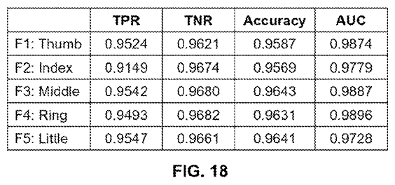

The table shown in FIG. 18 shows the classification performance results for individual fingers from the amputee. The data further demonstrate the neural decoder's exceptional capability with accuracy ranging from 95-96% and AUC ranging from 97-99%. Nevertheless, there are considerably more false-positives in the amputee prediction when compared to able participants. Some fingers, such as the amputee's index finger, are considerably less predictive than others. This disparity is often caused by a low signal-to-noise ratio in the nerve recordings. Alternative methods of practicing the claimed invention may add additional training sessions or train a specific model to control only a particular finger.

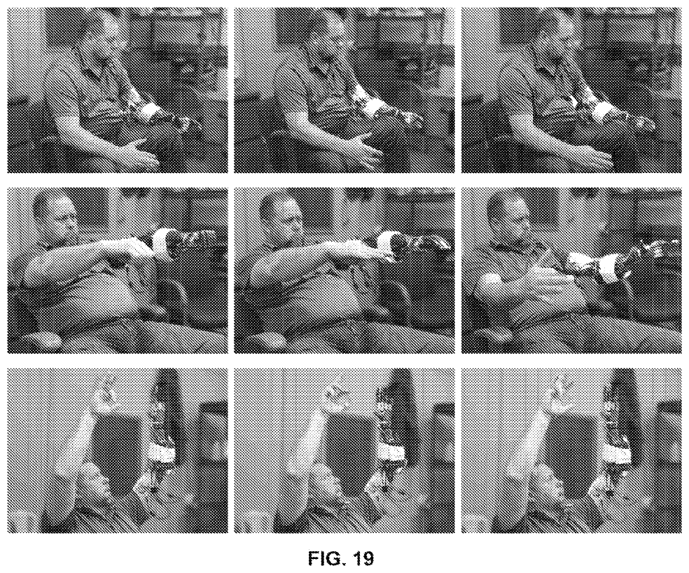

The series of photographic images of FIG. 19 shows an amputee testing the neuroprosthesis in a laboratory setting. Neural decoder models used in the training session shown were trained and optimized on a dataset that had been collected about two months earlier. The trained models were loaded onto the Jetson® Nano for inference-only operation. The prediction outputs were directly mapped to the movements of the prosthesis's digits. All data acquisition and processing operations were carried out in real-time by the Jetson® Nano. There were no wired nor wireless communications with any remote computer, and only the edge computer of the neuroprosthetic hand was utilized. The amputee used his able hand for the sole purpose of showing outside observers the amputee's intention of certain finger motions for purposes of comparison. The results demonstrated that the neuroprosthetic robotic hand accurately executed the operator's motor intent. The amputee also tested the neuroprosthetic robotic hand's robustness throughout various postures and motions, including holding the arms straight out in front and straight up, which did, however, introduce considerable EMG noises. The subject amputee reported a slight change in the system's responsiveness as the exercises progressed, but there was no significant degradation in motor decoding accuracy. It was also noted that the neuroprostheses' motorized fingers moved at a much slower rate than the amputee's able hand, and thus could not truly follow the operator's movement, but this limitation was investigated and determined to be a mechanical limitation of the commercial neuroprosthesis selected for this experiment. However, this constraint did not apply when the amputee was controlling a virtual hand e.g multi-joint dynamics rigid body simulations with contact (MuJoCo).

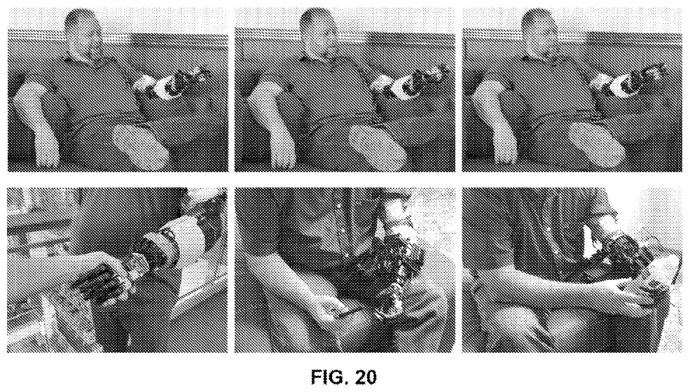

FIG. 20 shows the amputee testing the neuroprosthetic hand outside of the controlled environment of a laboratory, thereby subjecting the neuroprosthetic hand of the invention to real-world conditions. There are various additional noise sources in real-world settings that could affect the neuroprosthetic hand's systems and functions, for example WiFi, cellphones, electrical appliances, radio frequency emitting devices, etc. No evidence was seen suggesting any significant adverse impact on the system's performance during several hours of continuous operation under real world conditions.

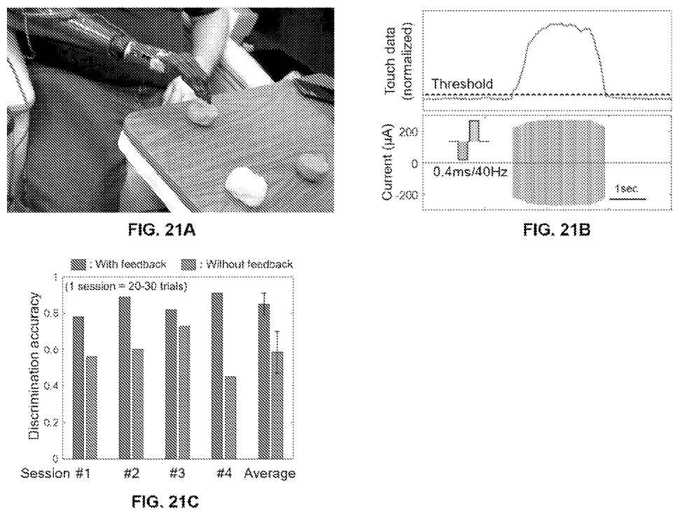

FIGS. 21A, 21B, and 21C taken together show quantitative representations of the results of a hand dexterity exercise using the neuroprosthetic hand of the invention. In FIG. 21A there is shown an object hardness discrimination exercise, which demonstrates the utility of having somatosensory feedback available to an amputee using the neuroprosthetic. The amputee was blindfolded and used the neuroprosthetic hand to tactily discriminate between three masses of different putties that each had differing degrees of material hardness, being prepared beforehand as being soft, medium, and hard relative to one another. Each putty was grasped and kneaded by the blindfolded amputee in random order. Four sessions were conducted, and in each session the amputee performed 20 to 30 trial manipulations of the materials, both with and without somatosensory feedback. These sessions and trials produced the stimulation pattern in FIG. 21B illustrating touch data 2100 and current flow 2102 as the amputee grasped and released the putty material. Touch sensor data was used to modulate the amplitude of the stimulation pulses. The stimulation pulses were only generated when they exceeded a defined threshold, and when the threshold was exceeded, a current of 200 microamps was generated and recorded, informing the process of modulation. FIG. 21C presents the results, indicating that having somatosensory feedback significantly increases the discrimination accuracy. Furthermore, the subject amputee reported that somatosensory feedback made the task greatly more intuitive and efficient. Without touch sensory capability, the subject amputee needed to use the entire prosthetic arm to “get a feel” for the object. He remarked that while it might be possible to match the accuracy with enough practice, doing so would be mentally exhaustive and would make the neuroprosthetic hand less effective and useful. This observation was consistent with past studies, which overwhelmingly suggests that artificial somatosensory feedback benefits the performance and confidence of an amputee using the neuroprosthetic hand in day-to-day living activity.

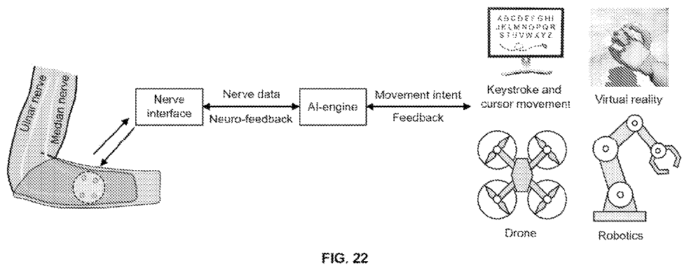

The flow chart of FIG. 22 illustrates alternative applications of the neuroprosthetic hand system 2200. Instead of sending the predicted movement intent to actuate a neuroprosthetic hand 2200, the nerve interface 2202 may engage in two-way communication of nerve data and neuro-feedback 2203 with AI engine 2204. AI engine 2204 can wirelessly transmit the results in terms of movement intent and device feedback 2206 to a remote controller to manipulate various devices and gadgets. These include a wide range of applications such as using a computer 2208 engaging in virtual reality 2210, flying a drone 2214, controlling a robot 2212, and so on. Such users are not limited to amputees but can be anyone who receives the nerve interface implant into their arm. The system of the invention allows a person to manipulate remote objects using only their thought in a true “telekinesis” manner.

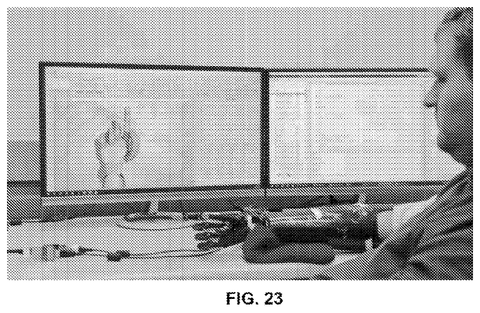

FIG. 23 shows an amputee using the nerve interface to play various video games with only his thought, though as explained above, such use is not limited to amputees. Each hand gesture was mapped to an individual keystroke in a computer keyboard. For example, thumb flexion was mapped to the “Space” bar, index flexion was mapped to the “W” key, fist/grip movement was mapped to the “F” key, etc. This allowed the amputee to perform various actions in video games like Raiden V (MOSS, 2016) and Far Cry 5 (Ubisoft Montreal and Ubisoft Toronto, 2018) with his thoughts like moving up/down/left/right and actuating primary/secondary acts on-screen. The experiment showed that a nerve interface of the invention could open up opportunities for other applications, beyond controlling a neuroprosthesis.

While the above description contains much specificity, these should not be construed as limitations on the scope of any embodiment, but as exemplifications of the presented embodiments thereof. Many other alternative embodiments and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention will not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are, unless otherwise stated, used in a generic and descriptive sense only and not for purposes of limitation, the scope of the invention therefore not being so limited. Moreover, the use of the terms first, second, etc. do not denote any order or hierarchy of importance, but rather the terms first, second, etc. are used to distinguish one element from another. Furthermore, the use of the terms a, an, etc. do not denote a limitation of quantity, but rather denote the presence of at least one of the referenced items.

While the invention has been described, exemplified, and illustrated in reference to certain preferred embodiments thereof, those skilled in the art will appreciate that various changes, modifications, and substitutions can be made therein without departing from the spirit and scope of the invention. It is intended, therefore that the invention be limited only by the scope of the claims which follow, and that such claims be interpreted as broadly as is reasonable.

What is claimed is:

1. A neuroprosthesis device, comprising:
a nerve interface;
an artificial intelligence engine
an artificial intelligence neural decoder run by said artificial intelligence engine; and
an electromechanical prosthetic limb.

2. The device as claimed in claim 1, wherein said nerve interface is comprised of a frequency shaping neural recorder and a redundant crossfire neural stimulator.

3. The device as claimed in claim 1, wherein said nerve interface is configured to establish bidirectional neural recording and neural stimulating communications with one or more selected residual peripheral nerves.

4. The device as claimed in claim 2, wherein said frequency-shaping neural recorder and said redundant crossfire neural stimulator are configured to establish said bidirectional recording and stimulating communications in near-simultaneous time.

5. The device as claimed in claim 4, wherein said frequency-shaping neural recorder and said redundant crossfire neural stimulator are configured to establish said bidirectional recording and stimulating communications simultaneously.

6. The device as claimed in claim 1, wherein said artificial intelligence neural decoder is configured to execute a deep learning architecture.

7. The device as claimed in claim 6, wherein said neural decoder using deep learning architecture gathers inputted nerve data from an amputee's movement intentions or motion intentions.

8. The device as claimed in claim 7, wherein said neural decoder using deep learning architecture gathers said inputted nerve data and translates said data into control of said electromechanical prosthetic limb.

9. The device as claimed in claim 8, wherein said electromechanical prosthetic limb is an electromechanical prosthetic hand.

10. The device as claimed in claim 9, where said artificial intelligence neural decoder is integrated into said electromechanical prosthetic hand.

11. The device as claimed in claim 10, wherein said electromechanical hand is a neuroprosthetic hand.

12. The device as claimed in claim 11, wherein said neuroprosthetic hand is configured to function as a phantom hand.

13. The device as claimed in claim 12, where said neuroprosthetic phantom hand comprises a prosthetic wrist having the ability to move through motions and a set of prosthetic fingers having the ability to move through motions, wherein said motions are directly controlled by said artificial intelligence engine.

14. The device as claimed in claim 13, where said artificial intelligence engine is configured so as to control said prosthetic wrist and said prosthetic fingers through movements and motions characterized as those of a natural wrist and natural fingers.

15. The device as claimed in claim 14, where said movements and motions are under intuitive control by a human wearing said device.

16. The device as claimed in claim 15, wherein said prosthetic fingers additionally comprise touch-sensitive sensors, said sensors configured to generate microstimulation patterns.

17. The device as claimed in claim 16, wherein said artificial intelligence decoder is configured to modulate said microstimulation patterns and provide somatosensory feedback to said prosthetic wrist and said prosthetic fingers.

18. The device as claimed in claim 17 configured so as to sequentially collect training data from said human, use said training data to train said artificial intelligence neural decoder, use said trained artificial intelligence neural decoder to create a trained model of movement through motions of said prosthetic wrist and said prosthetic fingers, and to deploy said trained model to generation of motion through movements of said prosthetic wrist and said prosthetic fingers.

19. The device as claimed in claim 1, where said nerve interface comprises a fully integrated bioelectronics circuit, comprising a plurality of fascicular microelectrodes implanted into selected nerve fibers of a peripheral nervous system, thereby connecting said selected nerve fibers with said artificial intelligence engine.

20. The device as claimed in claim 19, wherein said nerve interface comprises a plurality of microelectronic microchips configured so as to establish neural recording and neural stimulation simultaneously, said microelectronic microchips comprising at least one frequency-shaping amplifier configured to obtain ultra-low energy noise nerve signals, and to simultaneously suppress undesirable signal artifacts.

21. The device as claimed in claim 19, wherein said nerve interface comprises a high-precision analog to digital converter.

22. The device as claimed in claim 1, wherein said artificial intelligence engine comprises a standalone computer means.

23. The device as claimed in claim 1, wherein said artificial intelligence engine is configured to perform real-time motor decoding of outputs from said artificial intelligence neural decoder.

24. The device as claimed in claim 1, wherein said artificial intelligence engine comprises at least one system-on-chip mini-computer module, said computer module comprising an integrated central processing unit, a graphics processing unit, a random access memory, and a flash storage, and wherein said computer module is configured to deploy artificial intelligence software in an autonomous application.

25. The device as claimed in claim 24, wherein said graphics processing unit comprises a plurality of computer unified device architecture parallel processors, configured to run a deep learning library.

26. The device as claimed in claim 25, wherein said deep learning library is selected from the group consisting of TensorFlow, PyTorch, Caffe, Caffe 2, Chainer, CNTK, DSSTNE, DyNet, Genism, Gluon, Keras, Mxnet, Paddle, or BigDL.

27. The device as claimed in claim 24, where said artificial intelligence engine is optimized to require a minimum of electrical power required to run a selected deep learning library.

28. The device as claimed in claim 1, additionally comprising a rechargeable battery power supply.

29. The device as claimed in claim 9, where said hand comprises a hand controller comprised of a plurality of microcontrollers, a hand controller power supply, and a plurality of direct current motors for each digit of said hand, wherein said direct current motors are operated through said microcontrollers in response to decoded movement signals generated by deep-learning predictions calculated by said artificial intelligence engine.

30. The device as claimed in claim 1, additionally comprising an input/output unit configured to receive and transmit data, and a memory means configured to store data.
